(19) United States
(12) Patent Application Publication
(10) Pub. No.: US 2013/0150752 A1
(43) Pub. Date: Jun. 13, 2013

(54) Apparatus for Bone Aspiration

(71) Applicant: Karl W. Swann, San Antonio, TX (US)

(72) Inventor: Karl W. Swann, San Antonio, TX (US)

(21) Appl. No.: 13/323,269

(22) Filed: Dec. 12, 2011

(51) Int. Cl. A61B 10/02

(52) U.S. Cl. 600/567

(57) ABSTRACT

A bone marrow aspiration apparatus comprising a hollow; stylet removably positioned within said needle; a cylindrical threaded section; a depth gauge threadedly engagable with and movable longitudinally along the threaded section and having a proximal and distal ends, the proximal end being spaced a variable first distance from the end of the needle; a locking nut movable along the threaded section; a cap connection section having a fluid fitting therein; a cap engagable with the cap connection and engaged with the stylet; wherein the threaded section and the depth gauge define a stylet passage between the fluid fitting and the proximal end of the depth gauge; and wherein the variable first distance between the end of the hollow needle and the proximal end of the depth gauge corresponds to a position of the distal annular end of the depth gauge relative to the threaded section.

### CROSS REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of the Invention

The present invention relates to surgical devices. More specifically, the present invention is a bone aspiration device for removal of, inter alia, bone marrow from a patient's bone.

2. Description of the Related Art

In the process of bone marrow acquisition, the targeted bone is penetrated by a force, such as impaction, with a needle. When the needle has penetrated to a desired depth, a syringe is attached to the needle and bone marrow aspirated from the targeted. After the syringe contains a sufficient bone marrow sample, the needle can be disconnected and discarded.

### BRIEF SUMMARY OF THE INVENTION

The present invention is an improved aspiration apparatus for acquisition of bone marrow from a patient, and more frequently a patient's vertebrae. The present invention is low profile and allows easy penetration into the bone to aspirate the sample into a syringe.

The present invention comprises a hollow needle having a penetrating end and a distal end; a stylet having a penetrating end and a distal end, the stylet being removably positioned within said needle; a cylindrical threaded section; a depth gauge threadedly engagable with and movable longitudinally along the threaded section, the depth gauge having a proximal end and a distal annular end, the proximal end being spaced a variable first distance from the penetrating end of the hollow needle; a locking nut threadedly engagable with and movable longitudinally along the threaded section; a cap connection section having a fluid fitting therein; a cap having an interior space, the cap being engagable with the cap connection and engaged with the stylet, the cap having a distal end closed by a distal endwall, an open proximal end, and a sidewall extending between the distal end and the proximal end; wherein the threaded section and the depth gauge define a stylet passage between the fluid fitting of the cap connection and the proximal end of the depth gauge; and wherein the variable first distance between the penetrating end of the hollow needle and the proximal end of the depth gauge corresponds to a position of the distal annular end of the depth gauge relative to the threaded section.

### BRIEF DESCRIPTION OF THE SEVERAL VIEWS OF THE DRAWINGS

FIG. 1 is a top isometric view of the preferred embodiment of the present invention.

FIG. 2 is an assembly view of the embodiment shown in FIG. 1.

FIG. 3 is a reverse isometric view of the cap and sleeve shown in FIG. 2.

FIG. 4 is a side elevation of the embodiment shown in FIG. 1.

FIG. 5 is a second side elevation through line 5-5 of FIG. 4.

FIG. 6 is a sectional view of the preferred embodiment through line 6-6 of FIG. 4.

FIG. 7 is a sectional view of the preferred embodiment though line 7-7 of FIG. 5.

### DETAILED DESCRIPTION OF THE INVENTION

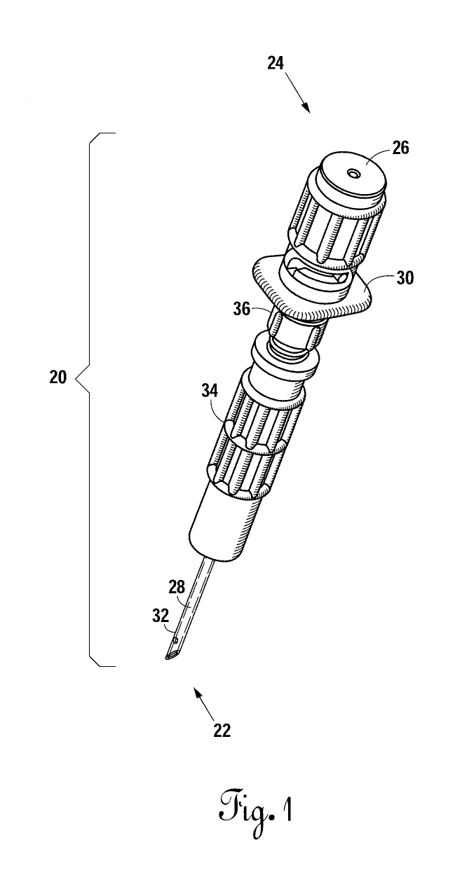

Referring to FIG. 1, the preferred embodiment 20 comprises a cap 26, a steel stylet 28, a sleeve 30, a needle 32, a depth guard 34, and a locking nut 36. Throughout this disclosure, the embodiment 20 is described with reference to a penetrating end 22 for penetrating through the skin and into the vertebrae of a patient to aspirate bone marrow, and an end 24 distal from the penetrating end 22. When elements of the invention are described herein, a “proximal end” is the end of the element closer to the penetrating end 22 as opposed to the “distal end.”

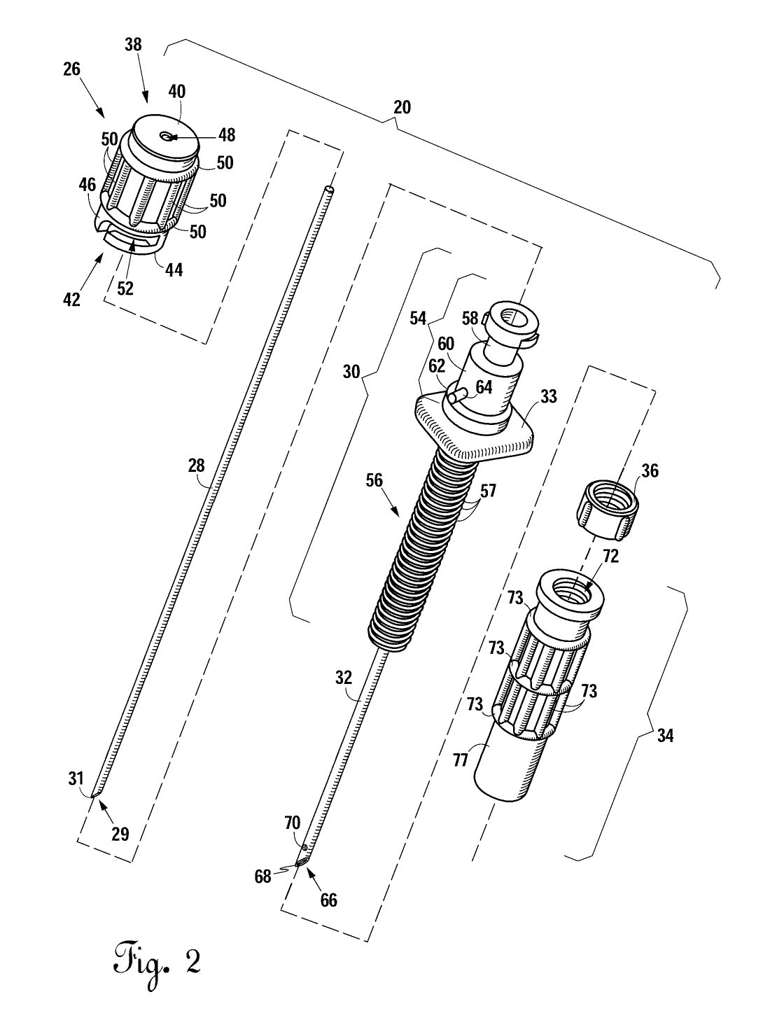

As shown in FIG. 2, the cap 26 is generally cylindrical and hollow, and has a distal end 38 closed by a distal endwall 40, an open proximal end 42 terminating in an annular proximal end wall 44, and a sidewall 46 extending between the distal and proximal endwalls 40, 44. A hole 48 is formed through the center of the distal endwall 40.

A plurality of longitudinal and circumferential ridges 50 extend from the sidewall 46 in the longitudinal and circumferential directions. A running slot 52 is formed in the sidewall 46 extending from the proximal endwall 44 and orientated circumferentially around the sidewall 44.

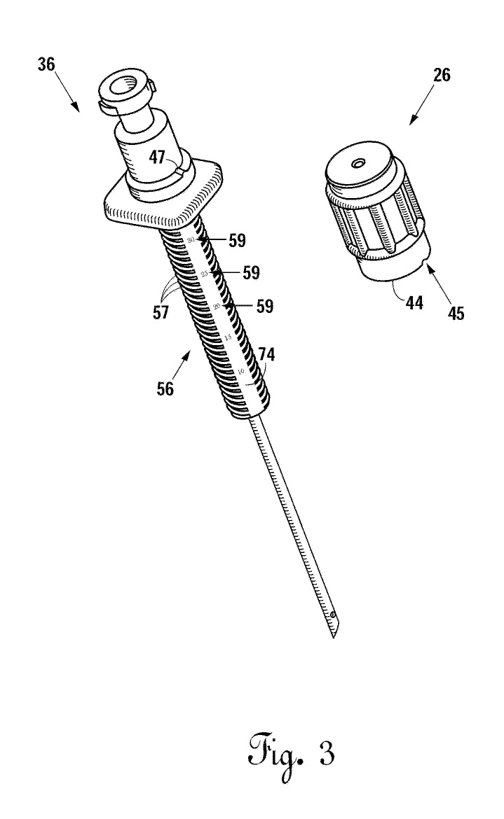

As shown in FIG. 3, a notch 45 is formed in the proximal endwall 44 of the cap 26. The notch 45 is sized and shaped to engage with an alignment tab 47 that inhibits rotation of the cap 26 relative to sleeve 30.

Referring back to FIG. 2, the sleeve 30 has a cap connection section 54 and a threaded section 56 having a series of external threads 57. A handle 33 is positioned between the threaded section 56 and the cap connection section 54.

The sharp steel trocar stylet 28 has a penetrating end 29 that terminates in a sharp point 31. The penetrating end 29 is angled relative to longitudinal axis of the stylet 28.

The cap connection section 54 comprises a distal annular section 58 having a first outer diameter, an intermediate annular section 60 having a second outer diameter, and a proximal annular section 62 having a third outer diameter. The first outer diameter is less than the second diameter, and the second outer diameter is less than the third outer diameter. First and second diameters are sized to fit within the interior space of the cap 26. A cylindrical boss 64 having a diameter sized to fit into the running slot 52 of the cap 26 extends radially from the intermediate annular portion 60.

The needle 32 extends from the threaded section 56 and has a penetrating end 66 partially defining the penetrating end 22 of the embodiment 20 (see FIG. 1). The penetrating end 66 is angled relative to the longitudinal axis of the needle 32 and terminates in a sharp point 68. The angle of the penetrating end 66 relative to the longitudinal axis corresponds to the angle of the penetrating end 29 of the stylet 28. The needle 32 has opposing lateral holes 70 spaced from the point 68.

The locking nut 36 has internal threads engagable with threaded section 56.

The depth guard 34 is generally tubular and has a sidewall 77 and an internally-threaded bore 72 engagable with the external threads 57 of the threaded section 56 of the sleeve 36. A plurality of longitudinal and circumferential ridges 73 extend from the sidewall 77 in the longitudinal and circumferential directions.

Referring to FIGS. 3-5, a planar surface 74 is formed in the threaded section 56 of the sleeve 30 such that the externals threads 57 are not continuous around at least a part of the threaded section 56 and are marked with distance markings 59 corresponding to a distance D₁ between the sharp point 68 of the needle 32 and the annular proximal end 90 of the depth gauge 34. For example, aligning the distal annular end 80 with a distance marking of X will correspondingly align the proximal annular end 90 of the depth gauge X units (e.g., millimeters) from the point 68 of the needle 32.

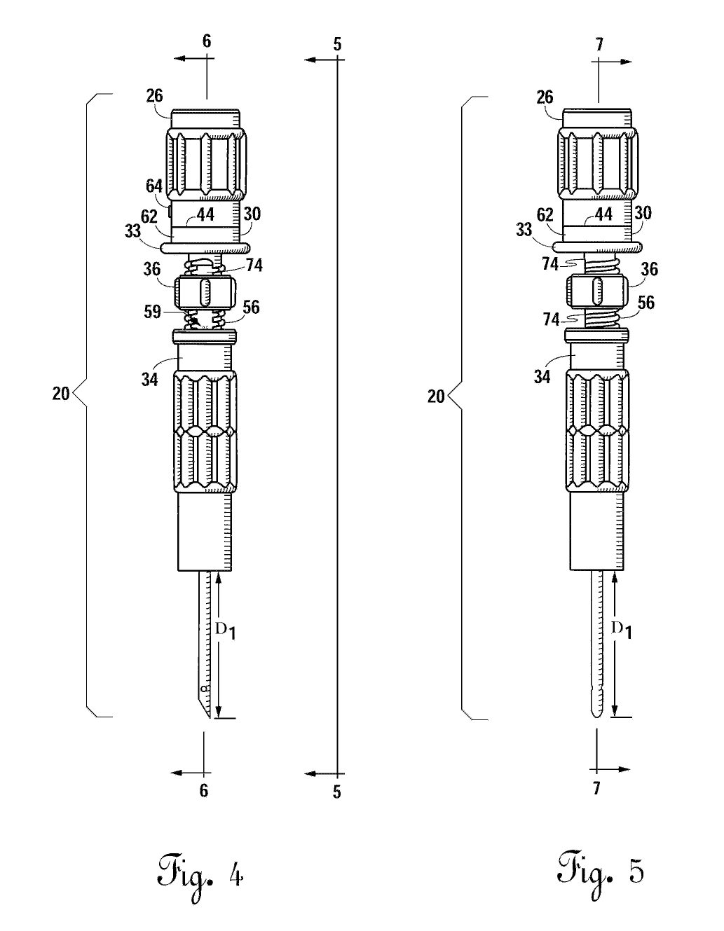

Referring just to FIGS. 4-5, the second endwall 44 of the cap 26 contacts the distal surface of the proximal annular section 62 of the cap connection section 54. The boss 64 is positioned within the running slot 52 (shown in FIG. 2). The locking nut 36 is longitudinally moveable between the handle 33 and the depth gauge 34.

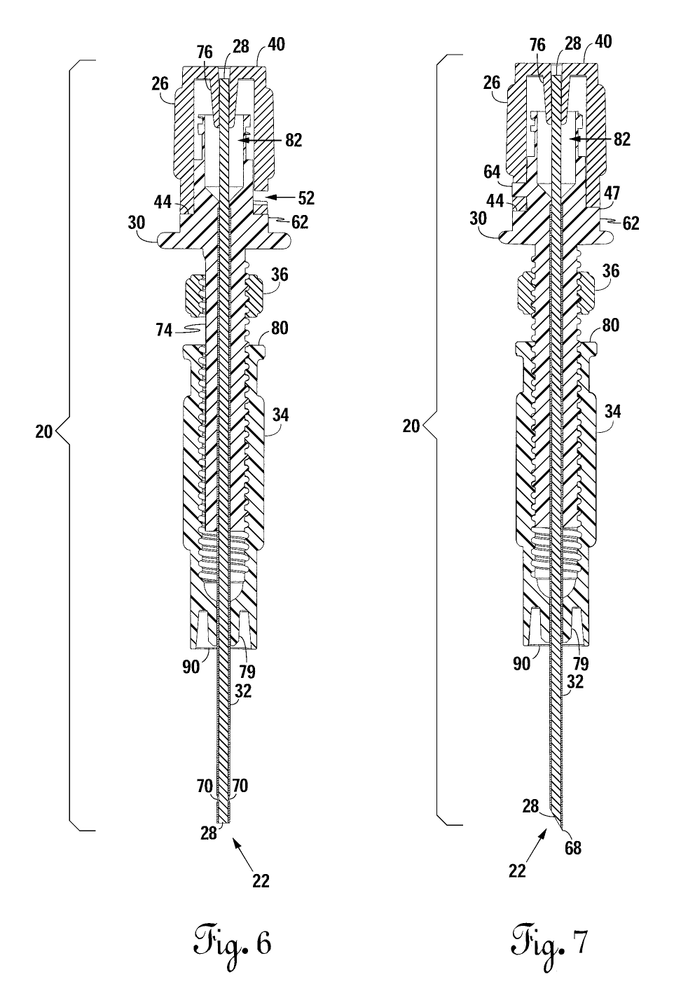

Referring to FIGS. 6-7, the cap 26 contains a tapered fluid fitting 76, such as a male Luer fitting, that extends from the distal endwall 40 into the interior space of the cap 26. The annular proximal endwall 44 contacts the distal surface of the proximal annular section 62 of the sleeve 30. The free end of the fluid fitting 76 extends into a female fluid fitting 82 formed in the sleeve 30. The depth gauge 34 is engaged with the threaded section 54 of the sleeve 30, and the locking nut 36 engaged with and moveable between the distal annular end 80 of the depth gauge 34 and the proximal surface of the handle 33. A male fluid fitting 79 is formed in the proximal end of the depth guard 34.

A passage is defined by the sleeve 30 and depth guard 34 for the holding the stylet 28 between the fluid fitting 76 of the cap 26 to the penetrating end 66 of the needle 32. The penetrating end of the stylet 28 and the penetrating end 66 of the needle 32 define the penetrating end 22 of the embodiment 20. In FIGS. 6-7, the needle 32 is shown as a separate element of the embodiment 20, but may be integrally formed with the sleeve 30.

Use of the embodiment is described with reference to FIGS. 6-7. The cap 26 is fastened over the distal end of the sleeve 30 with the proximal end 44 in contact with the proximal annular section 62. The boss 64 is positioned in the running slot 52 to prevent inadvertent removal of the cap 26. The stylet 28 is positioned in the fluid fitting 76 and through the stylet passage defined by the sleeve 30 and depth guard 34 to the penetrating end 66 of the needle 32. The ridges 50 (shown in FIG. 2) provide a convenient gripping surface for rotating the cap 26 with respect to the sleeve 30.

The depth guard 34 is threaded onto the threaded section 54 of the sleeve 30 to control the depth of the needle 32 into the patient. The depth limit is set by adjusting the depth guard 34 to the desired position on the threaded section 54 according to the distance markings 59 on the planar surface 74, causing the distance D₁ to correspond to the markings. Ridges 73 (shown in FIG. 2) provide a convenient gripping surface for rotating the depth gauge 34 with respect to the threaded section 56. The intended depth range for the needle 32 is from ten millimeters to thirty millimeters.

Once the desired depth is selected, the locking nut 36 is moved to a position adjacent to the distal annular end 80 of the depth guard 34 to prevent further movement of the depth guard 34 toward the handle 33, which would result in the depth limit inadvertently increasing and potentially endangering the patient when using the embodiment 20. The position of the locking nut 36 with respect to the depth guard 34 causes these elements to wedge the threads with opposing forces and prevent translation of the depth guard 34 on the threaded sleeve 30.

After the desired depth is selected and the locking nut 36 moved proximal to the depth gauge 34, the penetrating end 22 may then be inserted into the targeted vertebra (or other desired subject matter). The stylet 28 provides structural rigidity for the needle 32 during insertion. Insertion is limited to the distance D₁ between the sharp point 68 of the penetrating end 22 and the proximal surface 90 of the depth gauge 34.

After penetration of the penetrating end 22 to the desired depth, the cap 26 and stylet 28 may be removed. A syringe may then be inserted into the female fluid fitting 82 formed in the distal end 30 of the sleeve, and bone marrow aspirated through the needle 32. The lateral holes 70 through the sidewall of the needle 32 aids with aspiration. After the procedure, the embodiment 20 can be disconnected from the syringe and discarded.

The present invention is described in terms of a preferred illustrative embodiment of specifically-described bone aspiration apparatus. Those skilled in the art will recognize that yet other alternative embodiments of such an assembly can be used in carrying out the present invention. Other aspects, features, and advantages of the present invention may be obtained from a study of this disclosure and the drawings, along with the appended claims.

I claim:

1. An aspiration apparatus comprising:
a hollow needle having a penetrating end and a distal end;
a stylet having a penetrating end and a distal end, said stylet being removably positioned within said needle;
a cylindrical threaded section;
a depth gauge threadedly engagable with and movable longitudinally along said threaded section, said depth gauge having a proximal end and a distal annular end, said proximal end spaced a variable first distance from said penetrating end of said hollow needle;
a locking nut threadedly engagable with and movable longitudinally along said threaded section;
a cap connection section having a fluid fitting therein;
a cap having an interior space, said cap being engagable with said cap connection and engaged with said stylet, said cap having a distal end closed by a distal endwall, an open proximal end, and a sidewall extending between said distal end and said proximal end;
wherein said threaded section and said depth gauge define a stylet passage between said fluid fitting of said cap connection and the proximal end of said depth gauge; and
wherein said variable first distance between said penetrating end of said hollow needle and the proximal end of said depth gauge corresponds to a position of the distal annular end of said depth gauge relative to said threaded section.

2. The aspiration apparatus of claim 1 further comprising:
a boss extending radially from said cap connection section; and
wherein said cap includes a running slot positioned adjacent to said open proximal end of said cap, said boss selectively engagable with said running slot to inhibit removal of said cap from said cap connection section.

3. The aspiration apparatus of claim 1 further comprising a handle between said threaded section and said cap connection section.

4. The aspiration apparatus of claim 1 further comprising a male fluid fitting positioned at the proximal end of the depth gauge.

5. The aspiration apparatus of claim 1 wherein said needle further comprises at least one lateral hole spaced from said penetrating end of said needle operative.

6. The aspiration apparatus of claim 1 wherein said threaded section includes a planar surface having distances markings thereon, said distal annular end of said depth gauge being alignable with said distance marks.

7. The aspiration apparatus of claim 1 wherein said threaded section, a handle, and said cap connection compose a sleeve.

8. The aspiration apparatus of claim 1 further comprising a plurality of ridges formed on the sidewall of said cap.

9. The aspiration apparatus of claim 1 further comprising a plurality of ridges formed on the sidewall of said depth gauge.

10. The aspiration apparatus of claim 1 wherein said cap further comprises a fluid fitting extending from said distal endwall into said interior space of said cap.

11. The aspiration apparatus of claim 1 wherein said cap connection section includes a distal annular section having a first outer diameter, an intermediate annular section having a second outer diameter, and a proximal annular section having a third outer diameter, wherein said first outer diameter is less than said second outer diameter and said second outer diameter is less than said third outer diameter, and wherein said distal annular section and said intermediate annular section are sized to fit within the interior space of said cap.

12. The aspiration apparatus of claim 11 wherein said cap defines an alignment notch in said proximal annular end and said cap connection section further comprises an alignment tab sized to fit in said alignment notch to inhibit rotation of said cap relative to said cap connection section.
